SEQUENCE LISTING (22 sequences)

SEQ ID NO: 1 - protein, 1098 aa, Betacoronavirus Severe acute respiratory syndrome-related coronavirus
  source 1..1098: note = WT-extracellular domain
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQ

SEQ ID NO: 2 - protein, 1210 aa, Betacoronavirus Severe acute respiratory syndrome-related coronavirus D614G
  source 1..1210: note = D614G-extracellular domain
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 3 - protein, 1211 aa, Betacoronavirus Severe acute respiratory syndrome-related coronavirus B.1.1.7
  source 1..1211: note = B.1.1.7-extracellular domain
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKHFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 4 - protein, 1213 aa, Betacoronavirus Severe acute respiratory syndrome-related coronavirus B.1.351
  source 1..1213: note = B.1.351-extracellular domain
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 5 - protein, 1273 aa, Betacoronavirus Severe acute respiratory syndrome-related coronavirus B.1.617
  source 1..1273: note = B.1.617-extracellular domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTDWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1213 aa, Betacoronavirus Severe acute respiratory syndrome-related coronavirus P1
  source 1..1213: note = P1-extracellular domain
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 7 - protein, 1273 aa, Betacoronavirus Severe acute respiratory syndrome-related coronavirus B.1.617.1
  source 1..1273: note = B.1.617.1-extracellular domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1211 aa, Betacoronavirus Severe acute respiratory syndrome-related coronavirus B.1.617.2
  source 1..1211: note = B.1.617.2-extracellular domain
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 9 - protein, 1213 aa, Betacoronavirus Severe acute respiratory syndrome-related coronavirus B.1.617.3
  source 1..1213: note = B.1.617.3-extracellular domain
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 10 - protein, 222 aa, Betacoronavirus Severe acute respiratory syndrome-related coronavirus
  source 1..222: note = WT-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 11 - protein, 222 aa, Betacoronavirus Severe acute respiratory syndrome-related coronavirus B.1.1.7
  source 1..222: note = B.1.1.7-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 12 - protein, 222 aa, Betacoronavirus Severe acute respiratory syndrome-related coronavirus B.1.351
  source 1..222: note = B.1.351-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 13 - protein, 223 aa, Betacoronavirus Severe acute respiratory syndrome-related coronavirus B.1.617
  source 1..223: note = B.1.617-receptor binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNF

SEQ ID NO: 14 - protein, 222 aa, Betacoronavirus Severe acute respiratory syndrome-related coronavirus P1; identical to SEQ ID NO: 12
  source 1..222: note = P1-receptor binding domain

SEQ ID NO: 15 - protein, 223 aa, Betacoronavirus Severe acute respiratory syndrome-related coronavirus B.1.617.1; identical to SEQ ID NO: 13
  source 1..223: note = B.1.617.1-receptor binding domain

SEQ ID NO: 16 - protein, 222 aa, Betacoronavirus Severe acute respiratory syndrome-related coronavirus B.1.617.2
  source 1..222: note = B.1.617.2-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 17 - protein, 222 aa, Betacoronavirus Severe acute respiratory syndrome-related coronavirus B.1.617.3
  source 1..222: note = B.1.617.3-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 18 - protein, 419 aa, Betacoronavirus Severe acute respiratory syndrome-related coronavirus
  source 1..419: note = WT-N protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 19 - protein, 175 aa, Betacoronavirus Severe acute respiratory syndrome-related coronavirus
  source 1..175: note = WT-NSP3
IEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEK

SEQ ID NO: 20 - protein, 932 aa, Betacoronavirus Severe acute respiratory syndrome-related coronavirus
  source 1..932: note = WT-RdRp
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 21 - protein, 1210 aa, Betacoronavirus Severe acute respiratory syndrome-related coronavirus Omicron
  source 1..1210: note = Omicron-extracellular domain
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 22 - protein, 222 aa, Betacoronavirus Severe acute respiratory syndrome-related coronavirus Omicron
  source 1..222: note = Omicron-receptor binding domain
VQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

(19) United States
(12) Patent Application Publication
(10) Pub. No.: US 2023/0266309 A1
(43) Pub. Date: Aug. 24, 2023

(54) PROTEIN MICROARRAY, USE AND DETECTION METHOD THEREOF

(71) Applicant: NATIONAL CHENG KUNG UNIVERSITY, Tainan (TW)

(72) Inventor: Guan-da Syu, Tainan (TW)

(73) Assignee: NATIONAL CHENG KUNG UNIVERSITY, Tainan (TW)

(21) Appl. No.: 18/054,873

(22) Filed: Nov. 11, 2022

Related U.S. Application Data
(60) Provisional application No. 63/295,101, filed on Dec. 30, 2021.

(30) Foreign Application Priority Data
Sep. 27, 2022 (TW) 111136629

(51) Int. Cl. G01N 33/543; G01N 33/569; G01N 33/68

(52) U.S. Cl. CPC G01N 33/54373; G01N 33/6854; G01N 33/56983

(57) ABSTRACT

A protein microarray, a use thereof, and a detection method thereof are provided. The protein microarray includes a carrier with a protein array block on a surface thereof and at least two kinds of proteins immobilized on the protein array block. The at least two kinds of proteins include an extracellular region or a receptor binding region of the spike protein of a virus or a variant thereof. The protein microarray can detect the protective efficacy of vaccines, antibody drugs, or small molecule drugs against viral infection, or detect the immune response of an individual after vaccination or infection with a variant strain of the virus. The detection method of protein microarray includes the steps of adding the serum, a first fluorescent labeled human angiotensin converting enzyme 2, and a second fluorescent labeled anti-human immunoglobulin antibody to the protein microarray array and detecting optical signals.

### CROSS REFERENCE TO RELATED APPLICATION

The present application claims priority from U.S. Provisional Pat. Application Ser. No. 63/295,101, filed on Dec. 30, 2021: and Taiwan Patent Application No. 111136629 filed on Sep. 27, 2022 submitted to Intellectual Property Office, Ministry of Economic Affairs, R.O.C., with the invention titled “PROTEIN MICROARRAY, USE AND DETECTION METHOD THEREOF”, the entire contents of which are hereby incorporated by reference in this application.

### INCORPORATION BY REFERENCE OF SEQUENCE LISTING PROVIDED AS A XML FILE

The official copy of sequence listing is submitted concurrently with the specification as an XML file with a file name of TP221129-US-SEQUENCELIST.xml, a creation date of Oct. 3, 2022 and a size of 41,061 bytes. This sequence listing is part of the specification and is hereby incorporated in its entirely by reference herein.

### FIELD OF INVENTION

The present disclosure relates to the technical field of microarray, and particularly, to a protein microarray: the present disclosure also relates to the technical field of a use, and particularly, to a use of the protein microarray: and the present disclosure also relates to the technical field of a detection method, and particularly, to a detection method of the protein microarray.

### BACKGROUND OF INVENTION

In December 2019, it has been nearly three years since the cluster infection of severe special infectious pneumonia caused by combat the infection of severe acute respiratory syndrome coronavirus 2 (SARS-CoV-2) virus, also known as “coronavirus disease 2019” (COVID-19) was first reported in Wuhan City, mainland China. In order to combat the infection of COVID-19, at the end of 2020, the World Health Organization (WHO) has approved the COVID-19 vaccine for the emergency use. The COVID-19 vaccine comprises mRNA vaccines and recombinant viral vector vaccines. The mRNA vaccines comprise the BNT162b2 vaccine developed by American pharmaceutical company Pfizer Inc. and German biotechnology company BioNTech SE, and the mRNA-1273 vaccine developed by Moderna Inc. The recombinant viral vector vaccines comprise the JNJ-78436735 vaccine developed by Johnson & Johnson company, and the AZD1222 vaccine developed by the University of Oxford and AstraZeneca (AZ) company. Therefore, people around the world can be vaccinated against the infection of SARS-CoV-2 virus. In addition, when receiving the first dose, the second dose, and the third dose of the COVID-19 vaccine, people can choose the same brand of COVID-19 vaccine or mix and match different brands of COVID-19 vaccines.

Currently, the COVID-19 vaccines on the international market are all directed against the wild-type SARS-CoV-2 strains. However, due to the high mutation rate of mRNA viruses, the SARS-CoV-2 variants such as D614G, B.1.1.7 (i.e., a variant), B.1.351 (i.e., β variant), B.1.617, P1 (i.e., γ variant), B.1.617.1 (i.e., κ variant), B.1.617.2 (i.e., δ variant), B.1.617.3, and Omicron variant (i.e., BA.1/B.1.1.529) are prevalent in many countries of the world.

Due to the extremely protracted research and development of COVID-19 vaccines or drugs against SARS-CoV-2 virus, the speed of COVID-19 vaccine or drug development may not keep up with the mutation rate of SARS-CoV-2 virus. Therefore, the emergence of several SARS-CoV-2 variants has drawn attention to the protection ability of COVID-19 vaccines or drugs against SARS-CoV-2 variants.

The conventional technology mainly utilizes enzyme linked immunosorbent assay (ELISA) to analyze the titer of neutralizing antibodies against wild-type SARS-CoV-2 virus generated in an individual after inoculation with the COVID-19 vaccine, further evaluate the protection ability of the COVID-19 vaccine against wild-type SARS-CoV-2 virus infection. However, the ELISA can only detect the protection ability of the COVID-19 vaccine against wild-type SARS-CoV-2 virus infection and cannot detect the protection ability of the COVID-19 vaccine against wild-type SARS-CoV-2 virus and several SARS-CoV-2 variants infections in a single test.

In addition, due to the differences in the immune system among human individuals, the severity of the symptoms generated by different individuals after being infected with SARS-CoV-2 virus or SARS-CoV-2 variants may vary. Depending on the severity of the symptoms, the WHO has categorized the COVID-19 severity into mild/moderate, severe, and critical disease. Therefore, there are also significant differences in the immune characteristics of patients with mild/moderate, severe, and critical COVID-19. If physicians can understand the immune characteristics of patients with mild/moderate, severe, and critical COVID-19, it may help to assist in triage or preventive administration to reduce the risk of death of patients.

Therefore, development of a time-saving, low-cost, high-sensitivity, and high-throughput SARS-CoV-2 virus protein chip that ensures operator safety to evaluate the protection ability of COVID-19 vaccines or drugs against wild-type SARS-CoV-2 virus and several SARS-CoV-2 variants and analyze the immune characteristics of patients with mild/moderate, severe, and critical COVID-19 infected with SARS-CoV-2 virus or SARS-CoV-2 variant strains are urgent problems to be solved in the art.

### SUMMARY OF INVENTION

To solve the problems mentioned above, one object of the present disclosure is to provide a protein microarray. The object of detecting the protection ability of the COVID-19 vaccines, antibody drugs, or small molecule drugs against wild-type severe acute respiratory syndrome coronavirus 2 (SARS-CoV-2) virus and several SARS-CoV-2 variants infections in a single test may be achieved by immobilizing an extracellular domain or a receptor binding domain of a spike protein from the wild-type SARS-CoV-2 virus or variants thereof on a substrate of the protein microarray.

Another object of the present disclosure is to provide a use of a protein microarray. The object of quickly and accurately detecting an immune response in a subject after vaccination, or an immune response in a patient after being infected with the wild-type SARS-CoV-2 virus or variants thereof may be achieved by using the protein microarray for detection.

Still another object of the present disclosure is to provide a method for detecting an immune response in a subject. The objects of quickly and accurately detecting an immune response in a subject after vaccination, or an immune response in a patient after being infected with the wild-type SARS-CoV-2 virus or variants thereof, and categorizing the patient into the mild/moderate, severe, or critical case for preventive administration may be achieved by applying the serum or plasma of the patient to the protein microarray for detection.

In order to achieve the objects mentioned above, the present disclosure provides a protein microarray. The protein microarray may comprise a substrate and at least two proteins. A surface of the substrate comprises a plurality of protein array blocks and the at least two proteins are immobilized on each of the plurality of protein array blocks. The at least two proteins comprise an extracellular domain or a receptor binding domain of a spike protein from a virus, and an extracellular region or a receptor binding domain of a spike protein from a variant of the virus.

The extracellular domain of the spike protein of the virus comprises an amino acid sequence of SEQ ID NO: 1, and the receptor binding domain of the spike protein of the virus comprises an amino acid sequence of SEQ ID NO: 10.

The extracellular domain of the spike protein of the variant of the virus comprises any one of an amino acid sequence of SEQ ID NO: 2, SEQ ID NO: 3, SEQ ID NO: 4, SEQ ID NO: 5, SEQ ID NO: 6. SEQ ID NO: 7, SEQ ID NO: 8, SEQ ID NO: 9, or any combination thereof. The receptor binding domain of the spike protein of the variant of the virus comprises any one of an amino acid sequence of SEQ ID NO: 11, SEQ ID NO: 12. SEQ ID NO: 13, SEQ ID NO: 14, SEQ ID NO: 15, SEQ ID NO: 16, SEQ ID NO: 17. or any combination thereof.

In one embodiment, the virus comprises SARS-CoV-2 virus, and the variant of the virus comprises SARS-CoV-2 variant.

In one embodiment, the SARS-CoV-2 variant comprises SARS-CoV-2 D614G variant, SARS-CoV-2 B.1.1.7 variant, SARS-CoV-2 B.1.351 variant, SARS-CoV-2 B.1.617 variant, SARS-CoV-2 P1 variant, SARS-CoV-2 B.1.617.1 variant, SARS-CoV-2 B.1.617.2 variant, SARS-CoV-2 B.1.617.3 variant, SARS-CoV-2 B.1.1.529 variant, or any combination thereof.

In one embodiment, the at least two proteins further comprise a nucleocapsid protein, a nonstructural protein, an RNA-dependent RNA polymerase of the virus, or any combination thereof.

In one embodiment, the nucleocapsid protein has an amino acid sequence of SEQ ID NO: 18.

In one embodiment, the nonstructural protein has an amino acid sequence of SEQ ID NO: 19.

In one embodiment, the RNA-dependent RNA polymerase has an amino acid sequence of SEQ ID NO: 20.

The present disclosure further provides a use of the protein microarray described above. The protein microarray is used for in vitro detection of a protection efficacy of a vaccine, an antibody drug, or a small molecule drug against SARS-CoV-2 variant infection in a first subject; for in vitro detection of an immune response in a second subject after vaccination, or for in vitro detection of an immune response in a third subject after being infected with the SARS-CoV-2 variant.

In one embodiment, the vaccine is a COVID-19 vaccine.

In one embodiment, the first subject receives one dose of the COVID-19 vaccine or more than one dose of the COVID-19 vaccine. For example, the first subject receives a second dose, a third dose, a fourth dose, or a fifth dose of the COVID-19 vaccine.

In one embodiment, each of the more than one dose of the COVID-19 vaccine has the same brand name or different brand names.

In one embodiment, the COVID-19 vaccine comprises BNT162b2 vaccine (Pfizer-BioNTech), mRNA-1273 vaccine (Modema), AZD1222 vaccine (Oxford/AstraZeneca), or JNJ-78436735 vaccine (Johnson & Johnson).

In one embodiment, the antibody drug is a monoclonal antibody drug.

In one embodiment, the monoclonal antibody drug is a monoclonal antibody against a spike protein of a SARS-CoV-2 virus, a monoclonal antibody against a S1 domain of the spike protein of the SARS-CoV-2 virus, or a monoclonal antibody against a nucleocapsid protein of the SARS-CoV-2 virus.

In one embodiment, the small molecule drug comprises a receptor blocker.

In one embodiment, the small molecule drug comprises, but is not limited to Perindopril or Ramipril.

In one embodiment, the immune response comprises a human immunoglobulin G (IgG), a human immunoglobulin A (IgA), a human immunoglobulin M (IgM), or any combination thereof generated in the second subject and the third subject.

The present disclosure further provides a method for detecting an immune response in a subject, comprising the steps of:
providing the protein microarray described above;
adding a non-protein blocking reagent to the plurality of protein array blocks of the protein microarray for reacting 5 to 10 minutes to obtain a first protein microarray;
providing a to-be-tested sample from the subject, adding the to-be-tested sample to the first protein microarray for reacting 50 to 70 minutes, and then washing to obtain a second protein microarray;
provide a first fluorescently labeled angiotensin-converting enzyme 2 (ACE2) receptor on a human cell surface and a second fluorescently labeled anti-human immunoglobulin antibody, adding the first fluorescently labeled ACE2 receptor on the human cell surface and the second (fluorescently labeled anti-human immunoglobulin antibody to the second protein microarray, and reacting for 50 to 70 minutes followed by washing to obtain a third protein microarray; and
reading an optical signal generated from the third protein microarray by a signal reader to quantify the anti-human immunoglobulin antibody.

In one embodiment, the to-be-tested sample comprises a serum or a plasma of the subject.

In one embodiment, the anti-human immunoglobulin antibody comprises a human immunoglobulin G (IgG), a human immunoglobulin A (IgA), a human immunoglobulin M (IgM), or any combination thereof.

In one embodiment, a fluorescence used for the first fluorescently labeled ACE2 receptor comprise cyanine dye Cy3 or cyanine dye Cy5, and a fluorescence used for the second fluorescently labeled anti-human immunoglobulin antibody comprise cyanine dye Cy3 or cyanine dye Cy5. The fluorescence used for the first fluorescently labeled ACE2 receptor is different from the fluorescence used for the second fluorescently labeled anti-human immunoglobulin antibody.

The protein microarray of the present disclosure may quickly and accurately detect the protection ability of the COVID-19 vaccines, antibody drugs, or small molecule drugs against wild-type SARS-CoV-2 virus and several SARS-CoV-2 variants infections in a single test, and an immune response in a subject after vaccination by immobilizing the extracellular domain or the receptor binding domain of the spike protein from the wild-type SARS-CoV-2 virus or variants thereof on the substrate of the protein microarray. The protein microarray of the present disclosure may also quickly and accurately detect the immune response in the patient after being infected with the wild-type SARS-CoV-2 virus or variants thereof, and categorize the patient into the mild/moderate, severe, or critical case for preventive administration by applying the serum or plasma of the patient to the protein microarray for detection to reduce the risk of death of patients.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows the assay procedures to simultaneously qualify antibodies in serum or antibody drug against spike protein (S protein) (hereafter referred to as “anti-S mAb”) and surrogate neutralizing activities by using a first protein microarray of the present disclosure. The antibody amounts from either serum or anti-S mAb are quantified by Cy3-labeled anti-human antibody. The surrogate neutralizing activities are quantified by Cy5-labeled angiotensin-converting enzyme 2 (ACE2). The representative images from no antibody (a), low anti-S mAb (b), and high anti-S mAb (c) are listed. FIG. 1B shows the results of neutralizing activity of anti-S mAb by using the first protein microarray of the present disclosure. (a) Images of anti-S mAb with series dilution bound to the S proteins of the wild-type SARS-CoV-2 virus and SARS-CoV-2 variants. (b) Images of ACE2 receptor bound to the S proteins of the wild-type SARS-CoV-2 virus and SARS-CoV-2 variants in the presence or absence of anti-S mAb.

FIG. 1C shows the results of neutralizing activity of anti-S mAb by using the first protein microarray of the present disclosure. (a)-(i) Dual quantification of the ACE2 receptor and anti-S mAb bindings against S proteins of wild-type SARS-CoV-2 virus and SARS-CoV-2 variants, including D614G, B.1.1.7, B.1.351, P1, B.1.617, B.1.617.1, B.1.617.2, and B.1.617.3. The ACE2 binding is normalized by the antibody-free group for indicating the full attachment of viruses. Data are analyzed by two-way ANOVA with Dunnett’s multiple comparisons. *p<0.05, **p<0.01, ***p<0.005, and ****p<0.001, compared with zero dose of anti-S mAb.

FIG. 2 shows the results of high throughput detection of surrogate neutralizing activities against wild-type SARS-CoV-2 virus and SARS-CoV-2 variants in partial and fully vaccinated subjects by using a first protein microarray of the present disclosure. (A) Surrogate neutralizing activities against wild-type SARS-CoV-2 virus in partial vaccinated, fully vaccinated, and unvaccinated subjects. (B)-(I) Surrogate neutralizing activities against SARS-CoV-2 variants, including D614G, B.1.1.7, B.1.351, P1, B.1.617, B.1.617.1, B.1.617.2, and B.1.617.3. AZ, M, and UN refer to AZD1222, mRNA-1273, and unvaccinated, respectively. For AZ1, n=36; for AZ2, n=20; for M1, n=9; for M2, n=14, and for UN, n=33. Data are analyzed by one-way ANOVA followed by Tukey’s post-hoc tests, p<0.05 between different letters.

FIG. 3 shows the results of high throughput detection of serum IgG against wild-type SARS-CoV-2 virus and SARS-CoV-2 variants in partial and fully vaccinated subjects by using a first protein microarray of the present disclosure. Serum from partial vaccinated, fully vaccinated, and unvaccinated subjects are collected and analyzed for their IgG bindings. (A) IgG binding against wild-type SARS-CoV-2 virus in partial vaccinated, fully vaccinated, and unvaccinated subjects. (B)-(I) IgG binding against SARS-CoV-2 variants, including D614G, B.1.1.7, B.1.351, P1, B.1.617, B.1.617.1, B.1.617.2, and B.1.617.3. AZ, M, and UN refer to AZD1222, mRNA-1273. and unvaccinated, respectively. Data are analyzed by one-way ANOVA followed by Tukey’s post-hoc tests, p<0.05 between different letters.

FIG. 4 shows the results of high throughput detection of serum IgA against wild-type SARS-CoV-2 virus and SARS-CoV-2 variants in partial and fully vaccinated subjects by using a first protein microarray of the present disclosure. Serum from partial vaccinated, fully vaccinated, and unvaccinated subjects are collected and analyzed for their IgA bindings. (A) IgA binding against wild-type SARS-CoV-2 virus in partial vaccinated, fully vaccinated, and unvaccinated subjects. (B)-(I) IgA binding against SARS-CoV-2 variants, including D614G, B.1.1.7, B.1.351, P1, B.1.617, B.1.617.1, B.1.617.2, and B.1.617.3. AZ, M, and UN refer to AZD1222, mRNA-1273, and unvaccinated, respectively. Data are analyzed by one-way ANOVA followed by Tukey’s post-hoc tests, p<0.05 between different letters.

FIG. 5 shows the results of high throughput detection of serum IgM against wild-type SARS-CoV-2 virus and SARS-CoV-2 variants in partial and fully vaccinated subjects by using a first protein microarray of the present disclosure. Serum from partial vaccinated, fully vaccinated, and unvaccinated subjects are collected and analyzed for their IgM bindings. (A) IgM binding against wild-type SARS-CoV-2 virus in partial vaccinated, fully vaccinated, and unvaccinated subjects. (B)-(I) IgM binding against SARS-CoV-2 variants, including D614G, B.1.1.7, B.1.351, P1, B.1.617, B.1.617.1, B.1.617.2, and B.1.617.3. AZ, M, and UN refer to AZD1222, mRNA-1273, and unvaccinated, respectively. Data are analyzed by one-way ANOVA followed by Tukey’s post-hoc tests, p<0.05 between different letters.

FIG. 6 shows the results of neutralizing activity in healthy control (H), mild/moderate (M), severe (S), and critical (C) subjects by using a second protein microarray of the present disclosure. (A)-(I) Serums from H, M, S, and C groups are used to quantify the ACE2 binding against various spike proteins. The ACE2 binding is normalized by the serum-free group. Data are analyzed by one-way ANOVA with Tukey’s multiple comparisons. *p<0.05, **p<0.01, ***p<0.005, and ****p<0.001, compared with indicated groups. The number of subjects in the H, M, S, and C groups are 26, 25, 23, and 30, respectively.

FIG. 7 shows the results of quantification of serum IgG in healthy control (H), mild/moderate (M), severe (S), and critical (C) subjects by using a second protein microarray of the present disclosure. (A)-(I) Serums from H, M, S, and C groups are used to quantify the IgG binding against various spike proteins. (J), (K), and (L) Serums from H, M, S. and C groups are used to quantify the IgG binding against N protein, nonstructural protein (NSP3), and RNA-dependent RNA polymerase (RdRp) of wild-type SARS-CoV-2 virus. Data are analyzed by one-way ANOVA with Tukey’s multiple comparisons. *p<0.05, **p<0.01, ***p<0.005, and ****p<0.001, compared with indicated groups. The number of subjects in the H, M, S, and C groups are 26, 25, 23, and 30, respectively.

FIG. 8 shows the results of quantification of serum IgA in healthy control (H), mild/moderate (M), severe (S), and critical (C) subjects by using the second protein microarray of the present disclosure. (A)-(I) Serums from H, M, S, and C groups are used to quantify the IgA binding against various spike proteins. (J), (K), and (L) Serums from H, M, S, and C groups are used to quantify the IgA binding against N protein, NSP3, and RdRp of wild-type SARS-CoV-2 virus. Data are analyzed by one-way ANOVA with Tukey’s multiple comparisons. *p<0.05, **p<0.01, ***p<0.005, and ****p<0.001, compared with indicated groups. The number of subjects in the H, M, S, and C groups are 26, 25, 23, and 30, respectively.

FIG. 9 shows the results of quantification of serum IgM in healthy control (H), mild/moderate (M), severe (S), and critical (C) subjects by using the second protein microarray of the present disclosure. (A)-(I) Serums from H, M, S, and C groups are used to quantify the IgM binding against various spike proteins. (J), (K), and (L) Serums from H, M, S, and C groups are used to quantify the IgM binding against N protein, NSP3, and RdRp of wild-type SARS-CoV-2 virus. Data are analyzed by one-way ANOVA with Tukey’s multiple comparisons. *p<0.05, **p<0.01, ***p<0.005, and ****p<0.001, compared with indicated groups. The number of subjects in the H. M, S, and C groups are 26, 25, 23, and 30. respectively.

FIG. 10 shows the results of neutralizing activity for ACE2 inhibitors by using the second protein microarray of the present disclosure. (A)-(I) Two ACE2 inhibitors. e.g., Perindopril and Ramipril, are used to profile the binding of ACE2 against various spike proteins. The ACE2 binding is normalized by the inhibitor-free group. Data are analyzed by one-way ANOVA with Dunnett’s multiple comparisons. *p<0.05, **p<0.01, ***p<0.005, and ****p<0.001, compared with the zero dose of anti-S mAb.

FIG. 11 shows the results of neutralizing activity of receptor binding domain (RBD) in the COVID-19 vaccinated subjects (AZ×2: the subject received two doses of AZD1222 vaccine; M×2: the subject received two doses of mRNA-1273 vaccine; and AZ+M: the subject received one dose of AZD1222 vaccine and one dose of mRNA-1273 vaccine) by using a third protein microarray of the present disclosure. The neutralizing percentage is calculated based on the inhibition of ACE2 binding = 1-(ACE2 with plasma/ACE2 without plasma)×100%. (A) The neutralizing percentage of the plasma against the RBD of wild-type SARS-CoV-2 virus in the AZ×2, M×2, and AZ+M subjects. (B)-(F) The neutralizing percentage of the plasma against the RBD of SARS-CoV-2 B.1.1.7 variant (i.e., α variant), B.1.351 variant (i.e., β variant), P1 variant (i.e., γ variant), B.1.617.2 variant (i.e., δ variant), and B.1.1.529 variant (i.e., Omicron variant) in the AZ×2, M×2, and AZ+M subjects. Data are analyzed by the Kruskal-Wallis test followed by Dunn’s multiple comparisons. * p <0.05, ** p <0.01, and **** p<0.0001.

FIG. 12 shows the results of neutralizing activity of extracellular domain (ECD) in the COVID-19 vaccinated subjects (AZ×2: the subject received two doses of AZD1222 vaccine; M×2: the subject received two doses of mRNA-1273; and AZ+M: the subject received one dose of AZD1222 vaccine and one dose of mRNA-1273) by using the third protein microarray of the present disclosure. (A) The neutralizing percentage of the plasma against the ECD of wild-type SARS-CoV-2 virus in the AZ×2, M×2, and AZ+M subjects. (B)-(F) The neutralizing percentage of the plasma against the RBD of SARS-CoV-2 B.1.1.7 variant (i.e., α variant), B.1.351 variant (i.e., β variant), P1 variant (i.e., γ variant), B.1.617.2 variant (i.e., δ variant), and B.1.1.529 variant (i.e., Omicron variant) in the AZx2, M×2, and AZ+M subjects. Data are analyzed by the Kruskal-Wallis test followed by Dunn’s multiple comparisons. * p <0.05, ** p <0.01, and **** p<0.0001.

FIG. 13 shows the results of quantification of serum IgG, IgA, and IgM against RBD in the COVID-19 vaccinated subjects (AZ×2: the subject received two doses of AZD1222 vaccine; M×2: the subject received two doses of mRNA-1273; and AZ+M: the subject received one doses of AZD1222 vaccine and one dose of mRNA-1273) by using the third protein microarray of the present disclosure. (A) The binding antibody of the plasma against the RBD of wild-type SARS-CoV-2 virus in the AZ×2, M×2, and AZ+M subjects. (B)-(F) The binding antibody of the plasma against the RBD of SARS-CoV-2 B.1.1.7 variant (i.e., α variant), B.1.351 variant (i.e., β variant), P1 variant (i.e., γ variant), B.1.617.2 variant (i.e., δ variant), and B.1.1.529 variant (i.e., Omicron variant) in the AZ×2, M×2, and AZ+M subjects. Data are analyzed by the Kruskal-Wallis test followed by Dunn’s multiple comparisons. ****p<0.0001.

FIG. 14 shows the results of quantification of serum IgG, IgA, and IgM against ECD in the COVID-19 vaccinated subjects (AZ×2: the subject received two doses of AZD1222 vaccine: M×2: the subject received two doses of mRNA-1273; and AZ+M: the subject received one doses of AZD1222 vaccine and one dose of mRNA-1273) by using the third protein microarray of the present disclosure. (A) The binding antibody of the plasma against the ECD of wild-type SARS-CoV-2 virus in the AZ×2, M×2, and AZ+M subjects. (B)-(F) The binding antibody of the plasma against the RBD of SARS-CoV-2 B.1.1.7 variant (i.e., α variant), B.1.351 variant (i.e., β variant), P1 variant (i.e., γ variant), B.1.617.2 variant (i.e., δ variant), and B.1.1.529 variant (i.e., Omicron variant) in the AZ×2, M×2, and AZ+M subjects. Data are analyzed by the Kruskal-Wallis test followed by Dunn’s multiple comparisons. ****p<0.0001.

### DETAILED DESCRIPTION OF PREFERRED EMBODIMENTS

The following provides specific embodiments to illustrate the implementation of the present disclosure. A person having ordinary skill in the art can understand other advantages and effects of the present disclosure from the contents disclosed in the present specification. However, the exemplary embodiments disclosed in the present disclosure are only for illustrative purposes and should not be regarded as limiting the scope of the present disclosure. In other words, the present disclosure can also be implemented or applied by other different specific embodiments, and various details in the present specification can also be modified and changes based on different viewpoints and applications without departing from the concept of the present disclosure.

Unless otherwise indicated herein, the singular forms “one” and “the” used in the specification and the appended claims of the present disclosure include the plural. Unless otherwise indicated herein, the term “or” used in the specification and the appended claims of the present disclosure includes the meaning of “and/or”.

### Preparation Example 1: Preparation of a First SARS-CoV-2 Variant Protein Microarray (Hereinafter Referred to As “a First Protein Microarray”)

The extracellular domains (ECD) of spike proteins (hereinafter referred to as “S protein”) from wild-type SARS-CoV-2 virus and several SARS-CoV-2 variants with histidine tag (His-tag) listed in Table 1 are purchased from Sino Biological Inc. (Mainland China). The ECD is used as a biomarker for detecting virus infection. The ECD is composed of S1 subunit and S2 subunit. The S1 subunit comprises receptor binding domain (RBD), which may bind to angiotensin-converting enzyme 2 (ACE2) on a surface of human cell.

Each ECD of the S proteins is derived from the amino acid sequence set forth in SEQ ID NO: 1 from the wild-type SARS-CoV-2 virus, the amino acid sequence set forth in SEQ ID NO: 2 from the SARS-CoV-2 D614G variant, the amino acid sequence set forth in SEQ ID NO: 3 from the SARS-CoV-2 B.1.1.7 variant (i.e., α variant), the amino acid sequence set forth in SEQ ID NO: 4 from the SARS-CoV-2 B.1.351 variant (i.e., β variant), the amino acid sequence set forth in SEQ ID NO: 5 from the SARS-CoV-2 B.1.617 variant, the amino acid sequence set forth in SEQ ID NO: 6 from the SARS-CoV-2 P1 variant (i.e., γ variant), the amino acid sequence set forth in SEQ ID NO: 7 from the SARS-CoV-2 B.1.617.1 variant (i.e., κ variant), the amino acid sequence set forth in SEQ ID NO: 8 from the SARS-CoV-2 B.1.617.2 variant (i.e., δ variant), and the amino acid sequence set forth in SEQ ID NO: 9 from the SARS-CoV-2 B.1.617.3 variant.

The amino acid sequence set forth in SEQ ID NO: 1 from the wild-type SARS-CoV-2 virus comprises the amino acid sequence of receptor binding domain (RBD) set forth in SEQ ID NO: 10. The amino acid sequence set forth in SEQ ID NO: 3 from the SARS-CoV-2 B.1.1.7 variant (i.e., α variant) comprises the amino acid sequence of RBD set forth in SEQ ID NO: 11. The amino acid sequence set forth in SEQ ID NO: 4 from the SARS-CoV-2 B.1.351 variant (i.e., β variant) comprises the amino acid sequence of RBD set forth in SEQ ID NO: 12. The amino acid sequence set forth in SEQ ID NO: 5 from the SARS-CoV-2 B.1.617 variant comprises the amino acid sequence of RBD set forth in SEQ ID NO: 13. The amino acid sequence set forth in SEQ ID NO: 6 from the SARS-CoV-2 P1 variant (i.e., γ variant) comprises the amino acid sequence of RBD set forth in SEQ ID NO: 14. The amino acid sequence set forth in SEQ ID NO: 7 from the SARS-CoV-2 B.1.617.1 variant (i.e., κ variant) comprises the amino acid sequence of RBD set forth in SEQ ID NO: 15. The amino acid sequence set forth in SEQ ID NO: 8 from the SARS-CoV-2 B.1.617.2 variant (i.e., δ variant) comprises the amino acid sequence of RBD set forth in SEQ ID NO: 16. The amino acid sequence set forth in SEQ ID NO: 9 from the SARS-CoV-2 B.1.617.3 variant comprises the amino acid sequence of RBD set forth in SEQ ID NO: 17.

TABLE 1

Recombinant proteins of S proteins from wild-type SARS-CoV-2 virus and several SARS-CoV-2 variants with His-tag. Each S protein contains the ECD having RBD

| Virus | | Amino Acid Sequence | |
|---|---|---|---|
| | | ECD | RBD |
| Wild-type SARS-CoV-2 virus | SARS-CoV-2 virus | SEQ ID NO: 1 | SEQ ID NO: 10 |
| SARS-CoV-2 variant | D614G | SEQ ID NO: 2 | - |
| | B.1.1.7 (i.e., α variant) | SEQ ID NO: 3 | SEQ ID NO: 11 |
| | B.1.351 (i.e.. β variant) | SEQ ID NO: 4 | SEQ ID NO: 12 |
| | B.1.617 | SEQ ID NO: 5 | SEQ ID NO: 13 |
| | P1 (i.e., γ variant) | SEQ ID NO: 6 | SEQ ID NO: 14 |
| | B.1.617.1 (i.e., κ variant) | SEQ ID NO: 7 | SEQ ID NO: 15 |
| | B.1.617.2 (i.e., δ variant) | SEQ ID NO: 8 | SEQ ID NO: 16 |
| | B.1.617.3 | SEQ ID NO: 9 | SEQ ID NO: 17 |

After rinsing a glass slide with water, ethanol, acetone, and methanol in sequence, the glass slide is washed with 20% KOH solution for 2 hours at 65° C., and then washed with H₂SO₄/H₂O₂ solution in a volume ratio of 3:1 for 12 minutes to obtain a cleaned glass slide. The cleaned glass slide is then coated to obtain a surface-treated glass slide. The coating step comprises the steps of treating the cleaned glass slide with 2.5% 3-aminopropyl triethoxysilane dissolved in alcohol for 5 minutes, washing with pH 8.5, 0.5% glutaraldehyde dissolved in 0.05 M sodium borate solution for 16 hours, and then being dried. The surface-treated glass slide is stored in the vacuum-sealed bags at 4° C.

The proteins shown in Table 1 and the 11 samples of control group shown in Table 2 are spotted in three replicates on the surface-treated glass slide by using a microarray contact spotting system (CapitalBio SmartArrayer™ 136, Mainland China) to obtain the first protein microarray. The first protein microarray is stood overnight at room temperature before vacuum packaging and then stored at 4° C. or -80° C.

TABLE 2

Samples of control group and experimental group

| | |
|---|---|
| Control Group and Experimental Group | Chemical Compounds |
| Negative control group | Bovine serum albumin |
| | TBST buffer (Tris buffer and 0.1% Tween 20) |
| Positive control group | Cy3-landmark |
| | Cv5- landmark |
| Experimental group | Poly-L-lysine |
| | Protein A |
| | ACE2 |
| | Anti-human antibodies |
| | Anti-human-IgG |
| | Anti-human-IgA |
| | Anti-human-IgM |

### Preparation Example 2: Preparation of a Second SARS-CoV-2 Variant Protein Microarray (Hereinafter Referred to As “a Second Protein Microarray”)

The preparation method of the second protein microarray is similar to the preparation method of Preparation Example 1. The difference is that the nucleocapsid protein (hereinafter referred to as “N protein”) having an amino acid sequence set forth in SEQ ID NO: 18, the nonstructural protein (hereinafter referred to as “NSP3”) having an amino acid sequence set forth in SEQ ID NO: 19, and the RNA-dependent RNA polymerase (hereinafter referred to as “RdRp”) having an amino acid sequence set forth in SEQ ID NO: 20 from wild-type SARS-CoV-2 virus with His-tag listed in Table 3 purchased from Sino Biological Inc. (Mainland China) are further spotted on the second protein microarray.

TABLE 3

Recombinant proteins of N protein, NSP3, and RdRp from wild-type SARS-CoV-2 with His-tag

| | | |
|---|---|---|
| Wild-type SARS-CoV-2 virus | Protein | Amino Acid Sequence |
| | N Protein | SEQ ID NO: 18 |
| | NSP3 | SEQ ID NO: 19 |
| | RdRp | SEQ ID NO: 20 |

### Preparation Example 3: Preparation of a Third SARS-CoV-2 Variant Protein Microarray (Hereinafter Referred to As “a Third Protein Microarray”)

The preparation method of the third protein microarray is similar to the preparation method of Preparation Example 1. The difference is that the third protein microarray comprises the amino acid sequences shown in Table 4 which includes the amino acid sequence of receptor binding domain (RBD) set forth in SEQ ID NO: 10 from the amino acid SEQ ID NO: 1 of wild-type SARS-CoV-2 virus, the amino acid sequence of RBD set forth in SEQ ID NO: 11 from the amino acid SEQ ID NO: 3 of the SARS-CoV-2 B.1.1.7 variant (i.e., α variant), the amino acid sequence of RBD set forth in SEQ ID NO: 12 from the amino acid SEQ ID NO: 4 of the SARS-CoV-2 B.1.351 variant (i.e., β variant), the amino acid sequence of RBD set forth in SEQ ID NO: 14 from the amino acid SEQ ID NO: 6 of the SARS-CoV-2 P1 variant (i.e., γ variant), the amino acid sequence of RBD set forth in SEQ ID NO: 16 from the amino acid SEQ ID NO: 8 of the SARS-CoV-2 B.1.617.2 variant (i.e., δ variant), and the amino acid sequence of RBD set forth in SEQ ID NO: 22 from the amino acid SEQ ID NO: 21 of the SARS-CoV-2 B.1.1.529 variant (i.e., Omicron variant).

TABLE 4

Recombinant proteins of S proteins from wild-type SARS-CoV-2 virus and several SARS-CoV-2 variants with His-tag. Each S protein contains the ECD having RBD

| Virus | | Amino Acid Sequence | |
|---|---|---|---|
| | | ECD | RBD |
| WT SARS-CoV-2 virus | SARS-CoV-2 virus | SEQ ID NO: 1 | SEQ ID NO: 10 |
| SARS-CoV-2 variant | B.1.1.7 (i.e, α variant) | SEQ ID NO: 3 | SEQ ID NO: 11 |
| | B.1.351 (i.e, β variant) | SEQ ID NO: 4 | SEQ ID NO: 12 |
| | P1 (i.e, γ variant) | SEQ ID NO: 6 | SEQ ID NO: 14 |
| | B.1.617.2 (i.e, δ variant) | SEQ ID NO: 8 | SEQ ID NO: 16 |
| | B.1.1.529 (i.e, Omicron variant) | SEQ ID NO: 21 | SEQ ID NO: 22 |

### Example 1: Detection of the Ability of Antibody Drugs Against the S Protein of Wild-Type SARS-CoV-2 Virus and SARS-CoV-2 Variant by Using the First Protein Microarray

The binding of an antibody drug and ACE2 receptor is quantified by using the Cy3-labeled anti-human antibody and the Cy5-labeled ACE2 receptor on a human cell surface (Sino Biological Inc., Mainland China) in the present embodiment. In addition, the characteristic of using the antibody drug of a commercially available monoclonal antibody against the S1 domain of the S protein of SARS-CoV-2 virus (anti-S mAb) (purchased from Sino Biological Inc., Mainland China) to compete with the ACE2 receptor for binding to the S proteins of SARS-CoV-2 variants is used in the present disclosure to detect the specificity of the antibody drug against the S proteins of wild-type SARS-CoV-2 virus and SARS-CoV-2 variants.

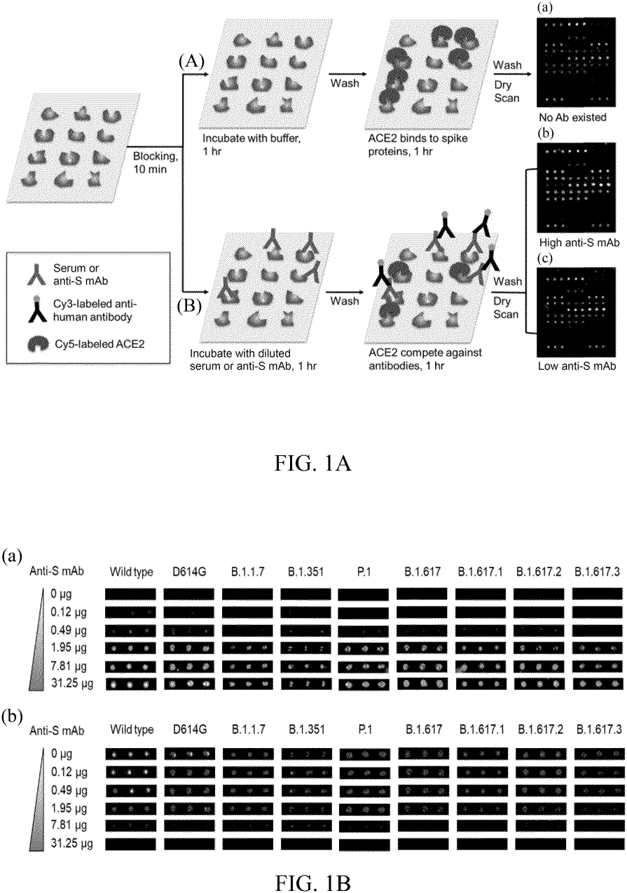

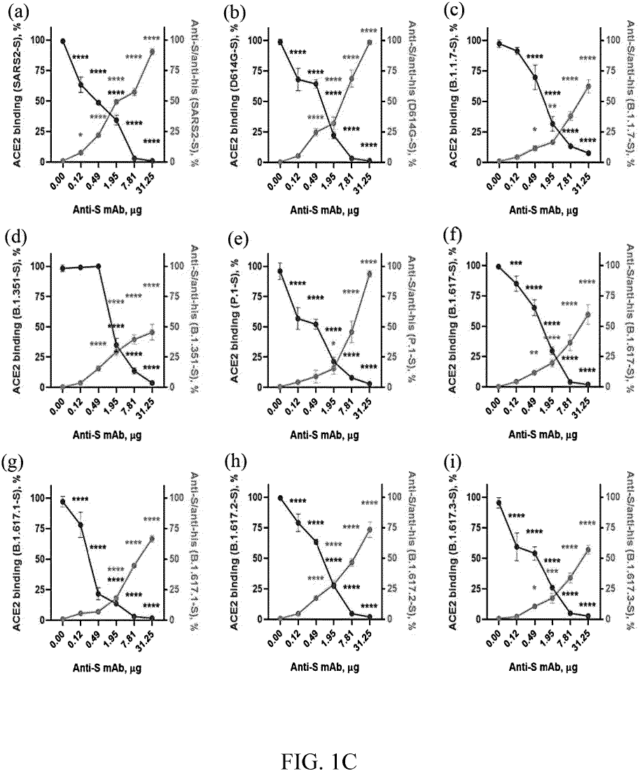

Referring to the process A of FIG. 1A, the first protein microarray is blocked with SuperBlock blocking reagent (Thermo Fisher Scientific, #37537) for 10 minutes, and then the buffer (TBST+1% BSA) is added for incubation for 1 hour. The first protein microarray is washed with TBST, and 50 µg Cy5-labeled human ACE2 (125 pg/mL) and Cy3-labeled anti-human IgG antibody are added for incubation for 1 hour, and then the first protein microarray is washed with TBST. Finally, the first protein microarray is dried and scanned for Cy3 and Cy5 signals.

Referring to the process B of FIG. 1A, the first protein microarray is blocked with SuperBlock blocking reagent (Thermo Fisher Scientific, #37537) for 10 minutes, and then the buffer (TBST+1% BSA) containing 31250 pg or 1953 pg antibody drug is added for incubation for 1 hour. The first protein microarray is washed with TBST, and 50 µg Cy5-labeled human ACE2 (125 pg/mL) and Cy3-labeled anti-human IgG antibody are added for incubation for 1 hour, and the first protein microarray is then washed with TBST. Finally, the first protein microarray is dried and scanned for Cy3 and Cy5 signals. The Cy3-labedled anti-human IgG antibody is used to detect the concentration of the antibody drug, and the Cy5-labedled human ACE2 is used to detect the neutralizing activity of the antibody drug against the S proteins of wild-type SARS-CoV-2 virus and SARS-CoV-2 variants.

The results show that in the process A of FIG. 1A, since the buffer that reacts with the first protein microarray does not contain the antibody that binds to the S protein of the SARS-CoV-2 variant, as shown in FIG. 1A (a), all of the detection results of the first protein microarray show red signals. In process B of FIG. 1A, since the antibody drug that reacts with the first protein microarray is an antibody that binds to the S protein of the SARS-CoV-2 variant, and the concentration of the antibody drug is 31,250 pg, as shown in FIG. 1A (b), the number of the green signal shown in the detection result of the first protein microarray is more than the number of the red signal. The result shows that a high neutralization potency is existed between the antibody drug and the wild-type SARS-CoV-2 virus, as well as between the antibody drug and the SARS-CoV-2 variant. In addition, as shown in FIG. 1A (c), since the concentration of the antibody drug reacted with the first protein microarray is 1,953 pg, the detection result of the first protein microarray shows low-brightness red signals or yellow signals. The result shows that a low neutralization potency is existed between the antibody drug and wild-type SARS-CoV-2 virus, as well as between the antibody drug and SARS-CoV-2 variant.

Referring to FIG. 1B and FIG. 1C. the results show that antibody drugs with serial dilution of 0.12 µg, 0.49 µg, 1.95 µg. 7.81 µg, and 31.25 µg may bind to the S proteins of wild-type SARS-CoV-2 and SARS-CoV-2 variants, and inhibit the binding of the ACE2 receptor to the S proteins of the wild-type SARS-CoV-2 and the SARS-CoV-2 variants in a dose-dependent manner. In addition, referring to FIG. 1C, Cy3 and Cy5 signals are respectively used to quantify the binding of antibody drugs and the ACE2 receptors to the S proteins of the wild-type SARS-CoV-2 virus and SARS-CoV-2 variants on the first protein microarray. The results show that the antibody drug and the ACE2 receptor may compete with each other for binding to the S proteins of the wild-type SARS-CoV-2 virus and the SARS-CoV-2 variants.

The above results show that the first protein microarray may effectively evaluate the ability of antibody drugs against the wild-type SARS-CoV-2 virus and SARS-CoV-2 variants. Therefore, the first protein microarray may facilitate to speed up the development of novel antibody drugs and other antiviral therapies against SARS-CoV-2 variants.

### Example 2: Detection of Specificity and Surrogate Neutralizing Activity Of Antibody Against the S Proteins of Wild-Type SARS-CoV-2 Virus and SARS-CoV-2 Variants in Subjects Partial or Fully Vaccinated Against COVID-19 by Using the First Protein Microarray

To examine the immune response of subjects partial or fully vaccinated against COVID-19, the serum is collected from unvaccinated subjects (UN), subjects vaccinated with one dose of AZD1222 (AZ1), subjects vaccinated with two doses of AZD1222 (AZ2), subjects vaccinated with one dose of mRNA-1273 vaccine (M1), and subjects vaccinated with two doses of mRNA-1273 vaccine (M2). The mean and standard deviation of the day after vaccination are 58.7 ± 9.0, 59.1 ± 24.9, 63.9 ± 28.6, and 57.1 ± 28.9 for AZ1, AZ2, M1, and M2, respectively.

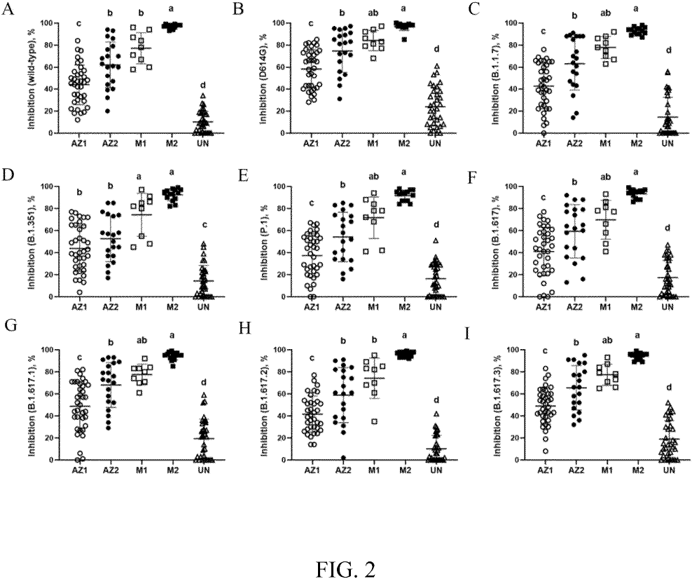

Referring to FIG. 2 (A) and (H), the results show that in the S proteins of wild-type SARS-CoV-2 virus and the SARS-CoV-2 B.1.617 variant, the surrogate neutralizing activity of antibody is highest in M2, followed by M1 or AZ2, followed by AZ1, and lowest in UN. Referring to FIG. 2 (B), (C), (E)-(G), and (I), the results show that in the S proteins of D614G, B.1.1.7, P1, B.1.617, B.1.6171 and B.1.617 of the SARS-CoV-2 variants, the surrogate neutralizing activities of antibodies are M2 ≥ M1 ≥ AZ2 > AZ1 > UN. Referring to FIG. 2 (D), the results show that in the S protein of the SARS-CoV-2 B.1.351 variant, the surrogate neutralizing activity of antibody is M2 = M1 > AZ2 = AZ1 > UN.

The above results show that although the surrogate neutralizing activities of antibodies among different vaccines are slightly different in the wild-type SARS-CoV-2 virus and the eight SARS-CoV-2 variants, they all share the similar tendency of higher in two doses than one dose, and higher in the mRNA-1273 vaccine than the AZD1222 vaccine. In addition, after being fully vaccinated, the M2 shows greater surrogate neutralizing activity than the AZ2 against the S proteins of wild-type SARS-CoV-2 virus and the eight SARS-CoV-2 variants. Therefore, the first protein microarray may facilitate to evaluate the protection ability of vaccines against SARS-CoV-2 variants.

### Example 3: Detection of IgG, IgA and IgM Levels Against the S Proteins of Wild-Type SARS-CoV-2 Virus and the Eight SARS-CoV-2 Variants in Subjects Partial or Fully Vaccinated Against COVID-19 by Using the First Protein Microarray

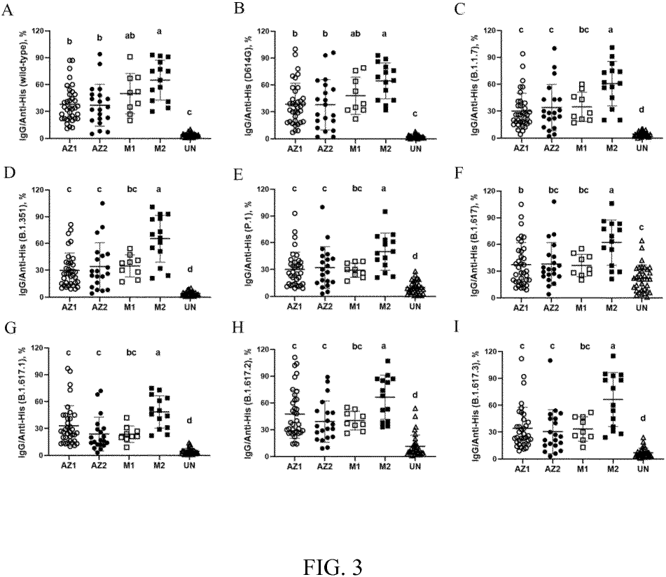

For IgG antibody, the IgG levels against the S proteins of wild-type SARS-CoV-2 virus and the eight SARS-CoV-2 variants in AZ1, AZ2, M1 and M2 subjects are detected by quantifying the IgG signals in the sera of AZ1, AZ2, M1 and M2 subjects. Referring to FIG. 3, the results show that except for the SARS-CoV-2 B.1.617.3 variant, the subjects vaccinated with one or two doses of AZD1222 vaccine may generate significant amounts of IgG antibodies against the S proteins of wild-type SARS-CoV-2 virus and the SARS-CoV-2 variants. However, the subjects vaccinated with two doses of AZD1222 may not generate more IgG antibodies than the subjects vaccinated with one dose of AZD1222. In contrast, the subjects vaccinated with two doses of mRNA-1273 vaccine may generate more IgG antibodies than the subjects vaccinated with one dose of mRNA-1273 vaccine. From the results, it can be seen that the surrogate neutralizing activity of the antibodies in the M2 subject is better than the surrogate neutralizing activity of the antibodies in the AZ2 subject.

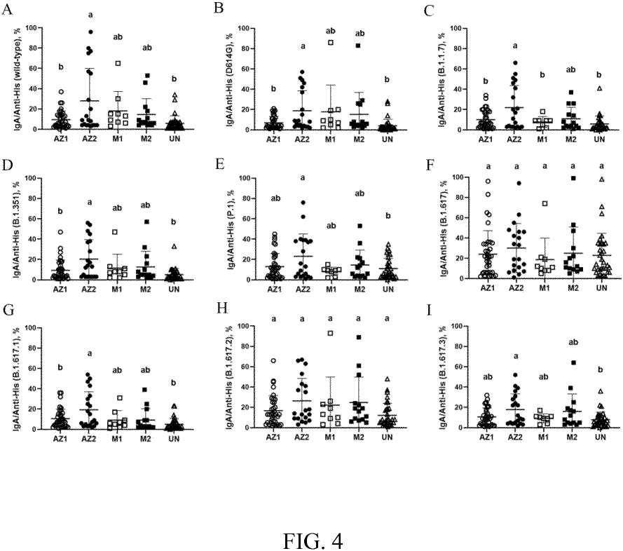

For IgA antibody, referring to FIG. 4, the results show that IgA antibodies are not generated in both M1 and M2 subjects. In contrast, except for the SARS-CoV-2 B.1.617 and B.1.617.2variants, two doses of AZD1222 vaccine may evoke a significant amount of IgA antibody against the S protein of wild-type SARS-CoV-2 virus and SARS-CoV-2 variants.

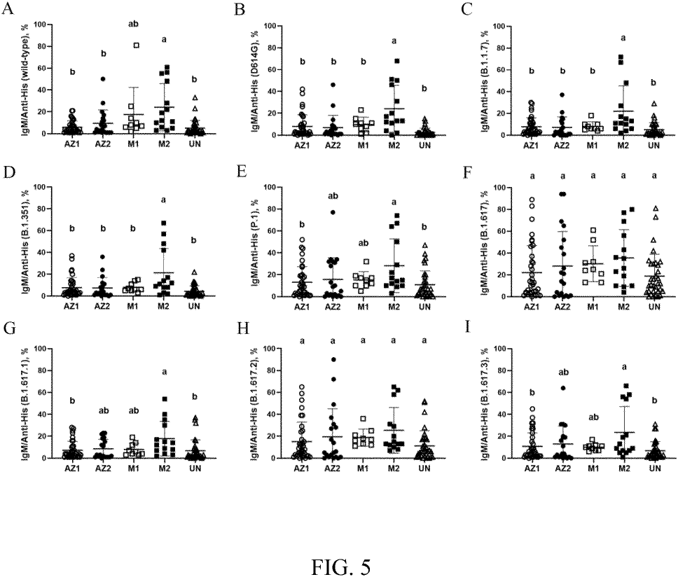

For IgM antibody, referring to FIG. 5, the results show that IgM antibodies are not generated in both AZ1 and AZ2 subjects. In contrast, except for the SARS-CoV-2 B.1.617 and B.1.617.2 variants, two doses of mRNA-1273 vaccine significantly increases the IgM level against the S protein of wild-type SARS-CoV-2 virus and SARS-CoV-2 variants.

From the above, it can be seen that the first protein microarray may effectively detect the amounts of IgG, IgA and IgM generated in the partial and fully vaccinated subjects against the S proteins of wild-type SARS-CoV-2 virus and SARS-CoV-2 variants.

### Example 4: Detection of the Antibody Response Against the S Proteins Of Wild-Type SARS-CoV-2 Virus and the Eight SARS-CoV-2 Variants in Healthy Subjects or Subjects with Different COVID-19 Severities by Using the Second Protein Microarray

The detection method of Example 4 is similar to the detection method of Example 1. The difference is that after blocking the second protein microarray with SuperBlock blocking reagent for 10 minutes, the buffer (TBST + 1% BSA) containing sera from a 50-fold diluted healthy subject (H) or sera from a 50-fold diluted SARS-CoV-2 virus or variant thereof-infected subject without COVID-19 vaccination is added for incubation for 1 hour. The amount of ACE2 receptor bound to the S proteins of SARS-CoV-2 variants on the second protein microarray is quantified by using Cy5-labeled human ACE2. The average data for blood sampling after symptom on set is 31 ± 16 days for mild/moderate (M) group, 27 ± 9 days for severe (S) group, and 10 ± 11 days for critical (C) group.

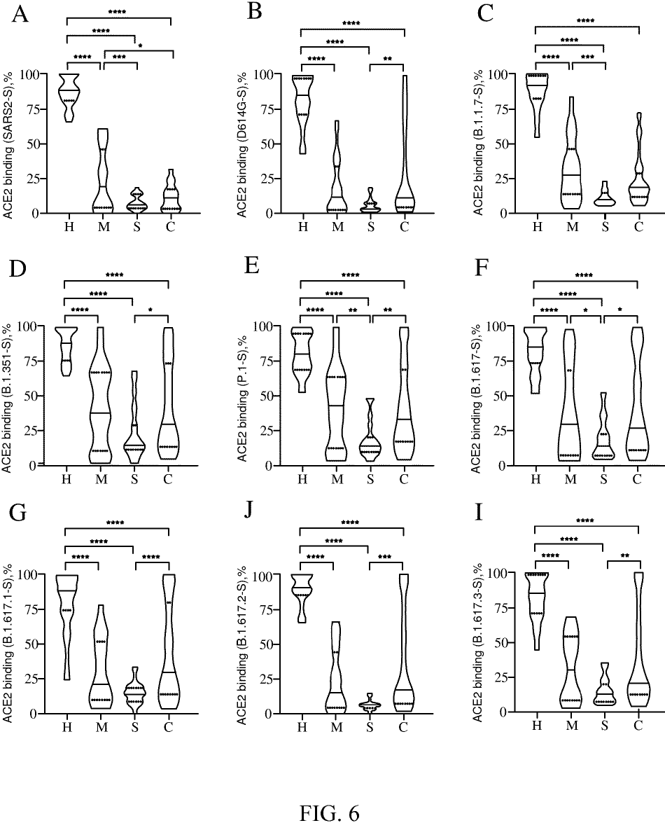

Referring to FIG. 6 (A)-(I), the results show that all the COVID-19 subjects show significant inhibition of the ACE2 binding compared with the H group. Referring to FIG. 6 (A), (C), (E), and (F), the results show that compared with mild/moderate (M) group, the severe (S) group shows more inhibition of the ACE2 bindings in wild-type SARS-CoV-2 virus and the SARS-CoV-2 B.1.1.7, P1, and B.1.617 variants. of, P1 and was high. Referring to FIG. 6 (B) and (D)-(I), the results show that compared with the critical (C) group, the severe (S) group shows more inhibition of ACE2 bindings in the SARS-CoV-2 D614G, B.1.351, P1, B.1.617, B.1.617.1, 8.1.617.2, and B.1.617.3 variants.

The above results clearly show that by detecting the degree of binding of the ACE2 receptor to the S protein of the SARS-CoV-2 variant strain in the second protein microarray, and the ability to detect healthy subjects or SARS-CoV-2 variant strains Antibody responses against the wild-type novel coronavirus and the S protein of SARS-CoV-2 variant strains in infected subjects, and effectively distinguish healthy subjects from subjects infected with SARS-CoV-2 variant strains with different symptoms.

### Example 5: Detection of IgG, IgA and IgM Levels Against the S Proteins of Wild-Type SARS-CoV-2 Virus and the Eight SARS-CoV-2 Variants and Against N Protein, NSP3, and RdRp of Wild-Type SARS-CoV-2 Virus in Healthy Subjects or Subjects with Different COVID-19 Severities by Using the Second Protein Microarray

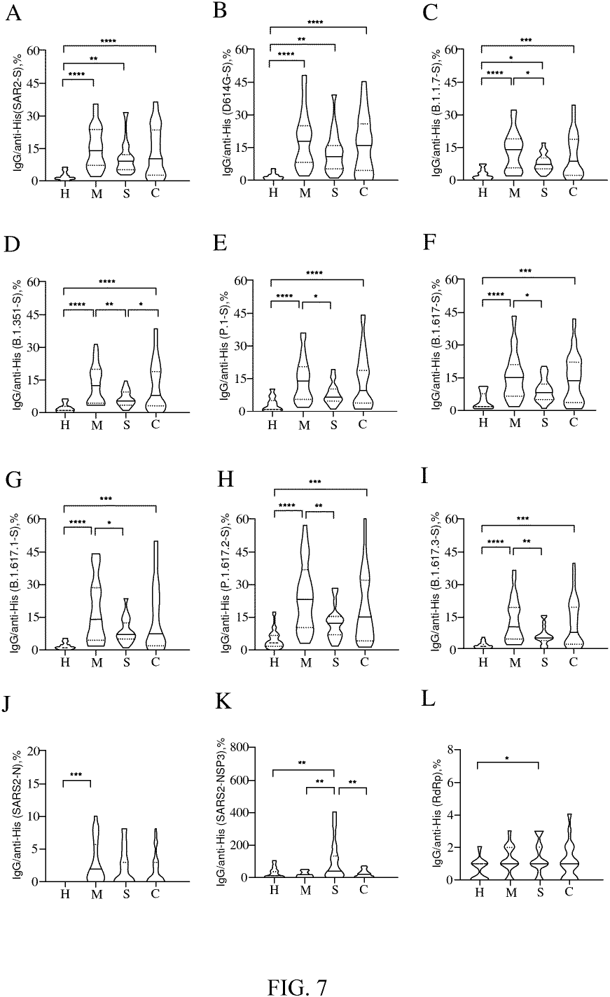

For IgG antibody. the IgG levels against the S proteins of wild-type SARS-CoV-2 virus and the eight SARS-CoV-2 variants in healthy (H), mild/moderate (M), severe (S), and critical (C) subjects are detected by quantifying the IgG signals in the sera of healthy (H), mild/moderate (M), and critical (C) subjects. Referring to FIG. 7 (C)-(I). the results show that compared with the mild/moderate (M) subjects, lower IgG levels against the S proteins of SARS-CoV-2 B.1.1.7, B.1.351, P1, B.1.617, B.1.617.1, B.1.617.2, and B.1.617.3 are found in the severe (S) subjects. Moreover, referring to FIG. 7 (J), the results show that only the mild/moderate (M) subjects may generate high levels of IgG antibodies against N protein. Therefore, the amount of IgG antibodies against N protein may be used as a marker to separate the severe (S) subjects and the critical (C) subjects. Furthermore, referring to FIG. 7 (K), the results show that compared to the healthy (H), mild/moderate (M), and critical (C) subjects, only the severe (S) subjects may generate the high level of IgG antibodies against NSP3. Therefore, the amounts of IgG antibodies against NSP3 may be used as a biomarker to distinguish the severe (S) subjects from the healthy (H), mild/moderate (M), and critical (C) subjects.

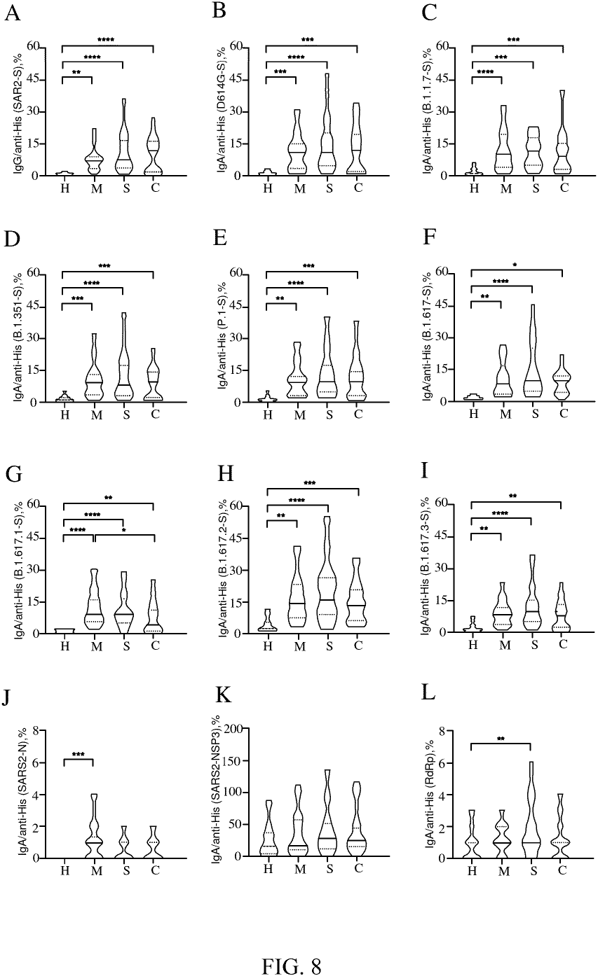

For IgA antibody, the IgA levels against the S proteins of wild-type SARS-CoV-2 virus and the eight SARS-CoV-2 variants in healthy (H), mild/moderate (M), severe (S), and critical (C) subjects are detected by quantifying the IgA signals in the sera of healthy (H), mild/moderate (M), and critical (C) subjects. Referring to FIG. 8 (A)-(I), the results show that in the SARS-COV-2 variants, IgA may effectively separate the COVID-19 patients with mild/moderate (M), severe (S), and critical (C) symptoms. Moreover, referring to FIG. 8 (F)-(H), for the SARS-CoV-2 B.1.617, B.1.617.1, and B.1.617.2variants, the IgA levels generated in the critical (C) subjects are lower than the IgA levels generated in the mild/moderate (M) subjects. Furthermore, referring to FIG. 8 (J), the results show that only the mild/moderate (M) subjects may generate high levels of IgA antibodies against N protein. Therefore, the amount of IgA antibodies against N protein may be used as a marker to separate the severe (S) subjects and the critical (C) subjects.

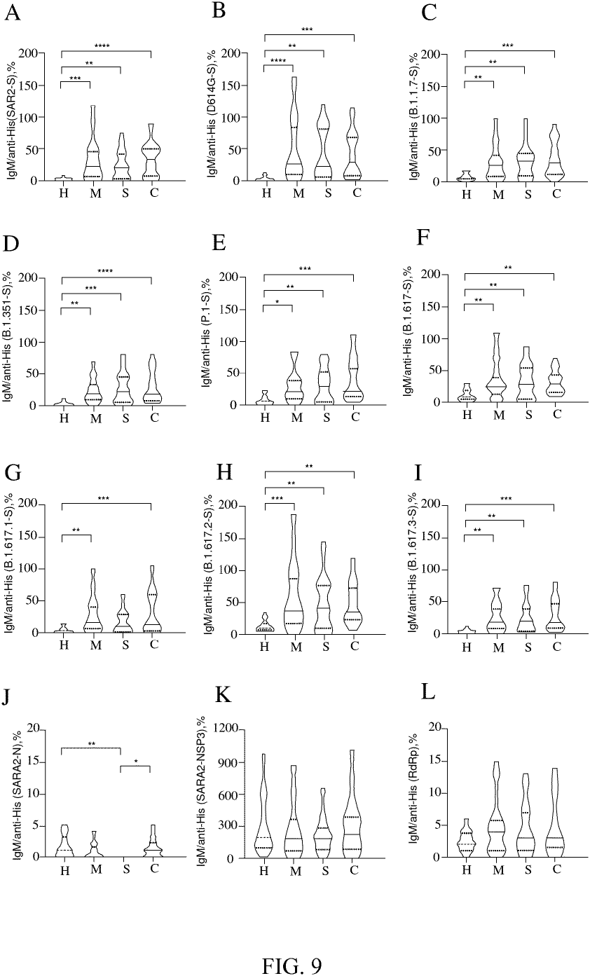

For IgM antibody, the IgM levels against the S proteins of wild-type SARS-CoV-2 virus and the eight SARS-CoV-2 variants in healthy (H), mild/moderate (M), severe (S), and critical (C) subjects are detected by quantifying the IgM signals in the sera of healthy (H), mild/moderate (M), and critical (C) subjects. Referring to FIG. 9 (A)-(I), the results show that the levels of IgM antibodies may effectively separate the COVID-19 patients with mild/moderate (M), severe (S), and critical (C) symptoms.

### Example 6: Detection of the Neutralizing Activities of Small Molecule Drugs Against Wild-Type SARS-CoV-2 Virus and the Eight SARS-CoV-2 Variants by Using the Second Protein Microarray

The detection method of Example 6 is similar to the detection method of Example 1. The difference is that the neutralizing activities of small molecule drugs against wild-type SARS-CoV-2 virus and the eight SARS-CoV-2 variants are detected by analyzing the inhibition degrees of Perindopril and Ramipril against the S proteins of wild-type SARS-CoV-2 virus and the SARS-CoV-2 D614G, B.1.1.7, B.1.351, P1, B.1.617, B.1.617.1, B.1.617.2, and B.1.617.3variants based on the binding properties of the ACE2 inhibitors, Perindopril and Ramipril, to the ACE2 receptors.

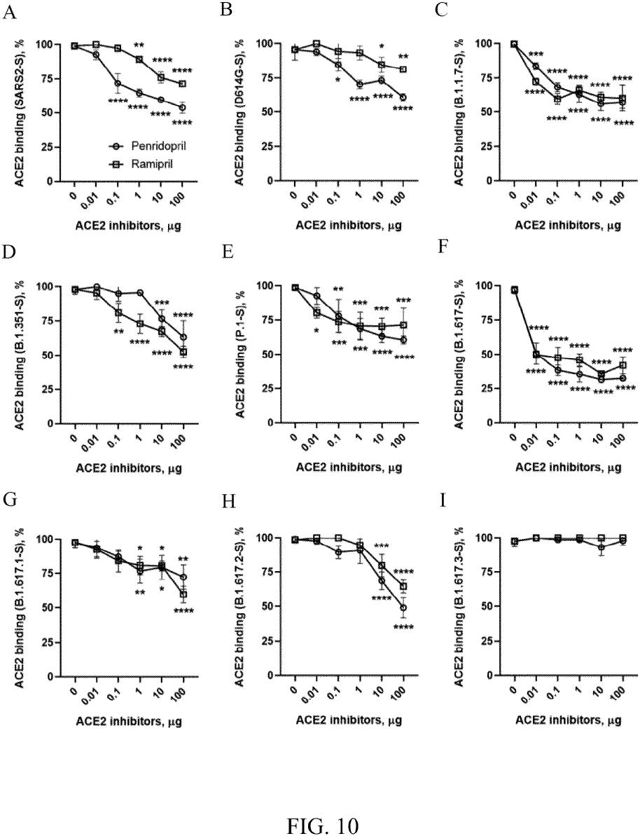

Referring to FIG. 10 (A)-(I), the results show that except for the SARS-CoV-2 B.1.617.3 variant, both of the two small molecule drugs, Perindopril and Ramipril, may dose-dependently decrease the ACE2 the binding to the S proteins of wild-type SARS-CoV-2 virus and the SARS-CoV-2 D614G, B.1.1.7. B.1.351. P1, B.1.617, B.1.617.1, and B.1.617.2 variants.

The above results show that the second protein microarray may effectively detect the neutralizing activities of small molecule drugs against wild-type SARS-CoV-2 virus and the SARS-CoV-2 variants.

### Example 7: Detection of the Neutralizing Activities of Antibodies in The Subjects Fully Vaccinated Against the S Proteins of Wild-Type SARS-CoV-2 Virus and the SARS-CoV-2 Variants by Using the Third Protein Microarray

In order to detect the immune responses of the subjects fully vaccinated against COVID-19, the plasma from the subjects received two doses of AZD1222 vaccine (AZx2), the subjects received two doses of mRNA-1273 vaccine (Mx2), and the subjects received one dose of AZD1222 vaccine and one dose of mRNA-1273 vaccine (AZ+M) are collected. The formula of inhibition of ACE2 binding=1-(ACE2 with plasma/ACE2 without plasma)×100% may calculate the neutralizing percentage of the plasma against the RBD of wild-type SARS-CoV-2 virus and SARS-CoV-2 B.1.1.7 variant (i.e., α variant), B.1.351 variant (i.e., β variant), P1 variant (i.e., γ variant), B.1.617.2 variant (i.e.. δ variant), and B.1.1.529 variant (i.e., Omicron variant).

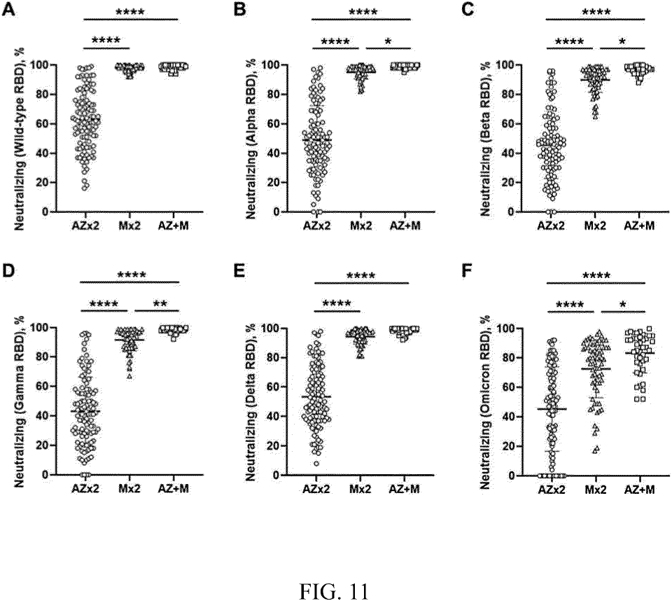

Referring to FIG. 11 (A)-(F), the results show that compared with the M×2 and AZ+M subjects, the lowest neutralizing activity of the antibody against the RBD of wild-type SARS-CoV-2 virus and SARS-CoV-2 variants is generated in the AZ×2 subjects. Moreover, referring to FIG. 11 (B)-(D) and (F), the results show that the highest neutralizing activity of the antibody against the RBD of wild-type SARS-CoV-2 virus and SARS-CoV-2 variants is generated in the AZ+M subjects.

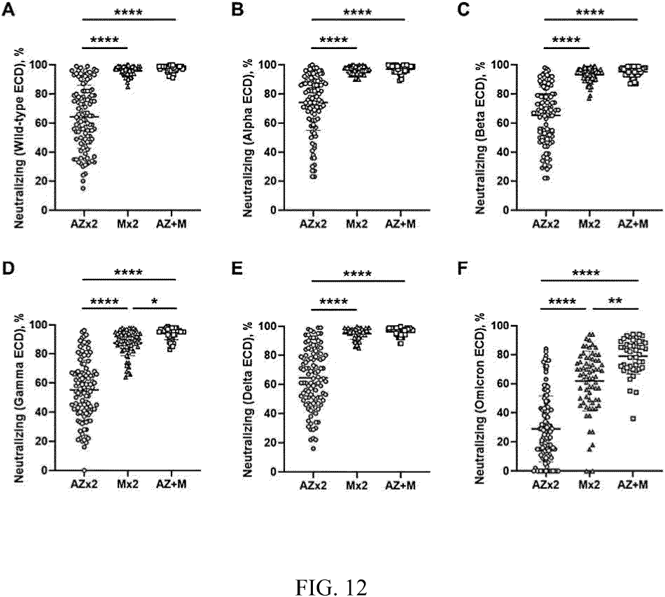

Referring to FIG. 12 (A)-(F), the results show that compared with the M×2 and AZ+M subjects, the lowest neutralizing activity of the antibody against the ECD of wild-type SARS-CoV-2 virus and SARS-CoV-2 variants is generated in the AZ×2 subjects. Moreover, referring to FIG. 12 (B)-(D) and (F), the results show that the highest neutralizing activity of the antibody against the ECD of wild-type SARS-CoV-2 virus and SARS-CoV-2 variants is generated in the AZ+M subjects.

The above results show that the third protein microarray may effectively evaluate the protection ability of vaccines against SARS-CoV-2 variants by detecting the neutralizing activity of the antibody against the RBD or ECD of wild-type SARS-CoV-2 virus and SARS-CoV-2 variants.

### Example 8: Detection of IgG, IgA and IgM Levels Against the S Proteins Of Wild-Type SARS-CoV-2 Virus and the SARS-CoV-2 Variants in Subjects Fully Vaccinated Against COVID-19 by Using the Third Protein Microarray

The detection method of this Example 8 is similar to the detection method of Example 3. The difference is that the IgG, IgA, and IgM levels in the subjects received two doses of AZD1222 vaccine (AZ×2), the subjects received two doses of mRNA-1273 vaccine (M×2), and the subjects received one dose of AZD1222 vaccine and one dose of mRNA-1273 vaccine (AZ+M) against RBD and ECD of wild-type SARS-CoV-2 virus and SARS-CoV-2 B.1.1.7 variant (i.e., α variant), B.1.351 variant (i.e., β variant), P1 variant (i.e., γ variant), B.1.617.2 variant (i.e., δ variant), and B.1.1.529 variant (i.e., Omicron variant) are detected.

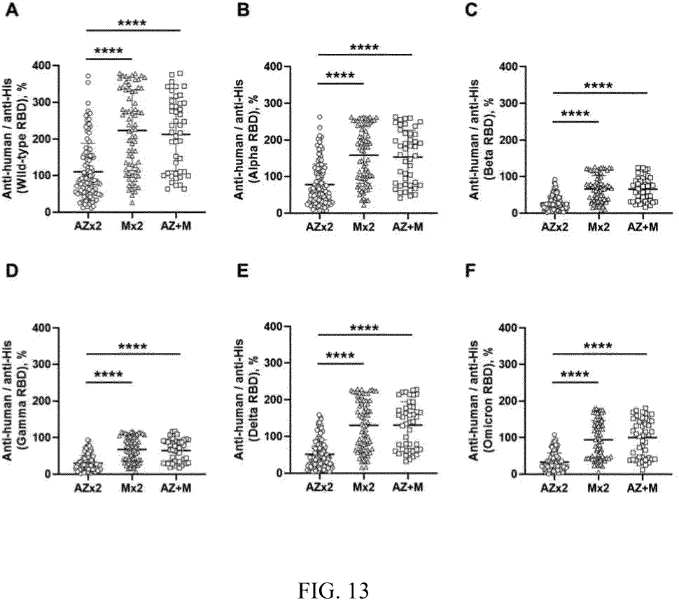

Referring to FIG. 13, the results show that compared with M×2 and AZ+M subjects, the lowest amounts of the antibodies against the RBD of wild-type SARS-CoV-2 virus and SARS-CoV-2 variants are generated in the AZ×2 subjects. In addition, there are no significant difference in the amounts of antibodies generated in M×2 and AZ+M subjects against RBD of wild-type SARS-CoV-2 virus and SARS-CoV-2 variants.

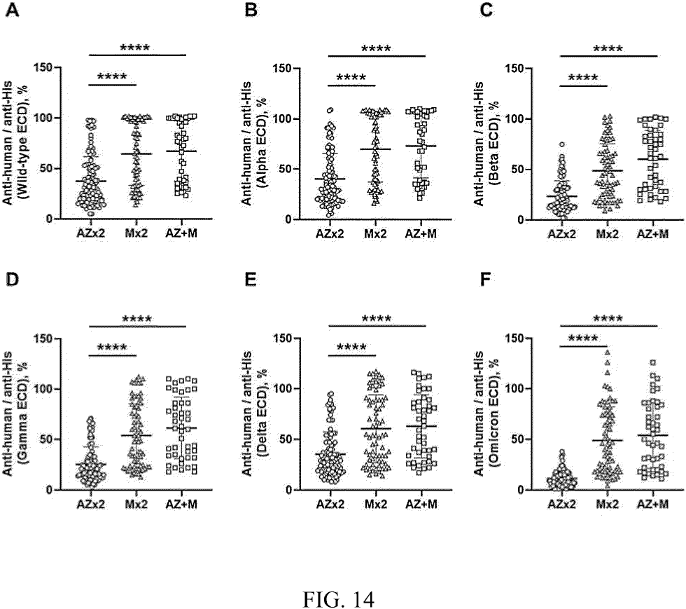

Referring to FIG. 14, the results show that compared with M×2 and AZ+M subjects, the lowest amounts of the antibodies against the ECD of wild-type SARS-CoV-2 virus and SARS-CoV-2 variants are generated in the AZ×2 subjects. In addition, there are no significant difference in the amounts of antibodies generated in M×2 and AZ+M subjects against ECD of wild-type SARS-CoV-2 virus and SARS-CoV-2 variants.

Based on the results described above, the protein microarray of the present disclosure may quickly and accurately detect the protection ability of the COVID-19 vaccines, antibody drugs, or small molecule drugs against wild-type SARS-CoV-2 virus and several SARS-CoV-2 variants infections in a single test, and an immune response in a subject after vaccination by immobilizing the extracellular domain or the receptor binding domain of the spike protein from the wild-type SARS-CoV-2 virus or variants thereof on the substrate of the protein microarray. The protein microarray of the present disclosure may also quickly and accurately detect the immune response in the patient after being infected with the wild-type SARS-CoV-2 virus or variants thereof, and categorize the patient into the mild/moderate, severe, or critical case for preventive administration by applying the serum or plasma of the patient to the protein microarray for detection to reduce the risk of death of patients.

The technical solutions of the present disclosure will be described clearly and completely in combined with the drawings of the present disclosure. Obviously, the described embodiments are only one part of the embodiments of the present disclosure, but not all of the embodiments. Based on the embodiments of the present disclosure, all other embodiments obtained by a person skilled in the art without making creative efforts fall within the claim scope of the present disclosure.

Although the present disclosure has been disclosed in preferred embodiments, it is not intended to limit the present disclosure. A person having ordinary skill in the art can make various changes and modifications without departing from the concept and scope of the present disclosure. Therefore, the claimed scope of the present disclosure shall be based on the scope defined by the attached claims of the patent disclosure.

What is claimed is:

1. A protein microarray, comprising:
a substrate comprising a plurality of protein array blocks on a surface of the substrate; and
at least two proteins immobilized on each of the plurality of protein array blocks, wherein the at least two proteins comprise an extracellular domain or a receptor binding domain of a spike protein from a virus, and an extracellular region or a receptor binding domain of a spike protein from a variant of the virus,
wherein the extracellular domain of the spike protein of the virus comprises an amino acid sequence of SEQ ID NO: 1;
the receptor binding domain of the spike protein of the virus comprises an amino acid sequence of SEQ ID NO: 10;
the extracellular domain of the spike protein of the variant of the virus comprises any one of an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or any combination thereof; and
the receptor binding domain of the spike protein of the variant of the virus comprises any one of an amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, or any combination thereof.

2. The protein microarray according to claim 1, wherein the virus comprises severe acute respiratory syndrome coronavirus 2 (SARS-CoV-2) virus, and the variant of the virus comprises SARS-CoV-2 variant, and wherein the SARS-CoV-2 variant comprises SARS-CoV-2 D614G variant, SARS-CoV-2 B.1.1.7 variant, SARS-CoV-2 B.1.351 variant, SARS-CoV-2 B.1.617 variant, SARS-CoV-2 P1 variant, SARS-CoV-2 B.1.617.1 variant, SARS-CoV-2 B.1.617.2 variant, SARS-CoV-2 B.1.617.3 variant, SARS-CoV-2 B.1.1.529 variant, or any combination thereof.

3. The protein microarray according to claim 1, wherein the at least two proteins further comprise a nucleocapsid protein, a nonstructural protein, or an RNA-dependent RNA polymerase of the virus.

4. The protein microarray according to claim 3, wherein the nucleocapsid protein has an amino acid sequence of SEQ ID NO: 18.

5. The protein microarray according to claim 3, wherein the nonstructural protein has an amino acid sequence of SEQ ID NO: 19.

6. The protein microarray according to claim 3, wherein the RNA-dependent RNA polymerase has an amino acid sequence of SEQ ID NO: 20.

7. A use of a protein microarray according to claim 1, wherein the protein microarray is used for in vitro detection of a protective efficacy of a vaccine, an antibody drug, or a small molecule drug against SARS-CoV-2 variant infection in a first subject; for in vitro detection of an immune response in a second subject after vaccination, or for in vitro detection of an immune response in a third subject after being infected with the SARS-CoV-2 variant.

8. The use according to claim 7, wherein the vaccine is a COVID-19 vaccine.

9. The use according to claim 8, wherein the COVID-19 vaccine comprises BNT162b2 vaccine (Pfizer-BioNTech), mRNA-1273 vaccine (Moderna), AZD1222 vaccine (Oxford/AstraZeneca), or JNJ-78436735 vaccine (Johnson & Johnson).

10. The use according to claim 8, wherein the first subject receives one dose of the COVID-19 vaccine or more than one dose of the COVID-19 vaccine.

11. The use according to claim 10, wherein each of the more than one dose of the COVID-19 vaccine has the same brand name or different brand names.

12. The use according to claim 7, wherein the antibody drug is a monoclonal antibody drug.

13. The use according to claim 12, wherein the monoclonal antibody drug is a monoclonal antibody against a spike protein of a SARS-CoV-2 virus, a monoclonal antibody against a S1 domain of the spike protein of the SARS-CoV-2 virus, or a monoclonal antibody against a nucleocapsid protein of the SARS-CoV-2 virus.

14. The use according to claim 7, wherein the small molecule drug comprises a receptor blocker.

15. The use according to claim 7, wherein the small molecule drug comprises Perindopril or Ramipril.

16. The use according to claim 7, wherein the immune response comprises a human immunoglobulin G (IgG), a human immunoglobulin A (IgA), a human immunoglobulin M (IgM), or any combination thereof generated in the second subject and the third subject.

17. A method for detecting an immune response in a subject, comprising the steps of:
providing a protein microarray of claim 1;
adding a non-protein blocking reagent to the plurality of protein array blocks of the protein microarray for reaction to obtain a first protein microarray;
providing a to-be-tested sample from the subject, adding the to-be-tested sample to the first protein microarray for reaction, and then washing to obtain a second protein microarray;
provide a first fluorescently labeled angiotensin-converting enzyme 2 (ACE2) receptor on a human cell surface and a second fluorescently labeled anti-human immunoglobulin antibody, adding the first fluorescently labeled ACE2 receptor on the human cell surface and the second fluorescently labeled anti-human immunoglobulin antibody to the second protein microarray, and reacting for 50 to 70 minutes followed by washing to obtain a third protein microarray; and
reading an optical signal generated from the third protein microarray by a signal reader to quantify the anti-human immunoglobulin antibody.

18. The method according to any one of claim 17, wherein the anti-human immunoglobulin antibody comprises a human immunoglobulin G (IgG), a human immunoglobulin A (IgA), a human immunoglobulin M (IgM), or any combination thereof.

19. The method according to any one of claim 17, wherein a fluorescence used for the first fluorescently labeled ACE2 receptor comprise cyanine dye Cy3 or cyanine dye Cy5, and a fluorescence used for the second fluorescently labeled anti-human immunoglobulin antibody comprise cyanine dye Cy3 or cyanine dye Cy5, and wherein the fluorescence used for the first fluorescently labeled human angiotensin-converting enzyme 2 receptor is different from the fluorescence used for the second fluorescently labeled anti-human immunoglobulin antibody.

20. The method according to claim 17, wherein the to-be-tested sample comprises a serum or a plasma of the subject.
